(19) United States
(12) Patent Application Publication
(10) Pub. No.: US 2026/0114758 A1
(43) Pub. Date: Apr. 30, 2026

(54) PAPER-BASED WEARABLE PATCHES FOR REAL-TIME, QUANTITATIVE LACTATE MONITORING

(71) Applicant: Trustees of Tuffs College, Medford, MA (US)

(72) Inventors: Fiorenzo G. Omenetto, Lexington, MA (US); Elisabetta Ruggeri, Cambridge, MA (US); Giuseppina Matzeu, Medford, MA (US)

(21) Appl. No.: 19/293,617

(22) Filed: Aug. 7, 2025

Related U.S. Application Data
(63) Continuation of application No. PCT/US24/14999, filed on Feb. 8, 2024.
(60) Provisional application No. 63/483,959, filed on Feb. 8, 2023.
(60) Provisional application No. 63/512,534, filed on Jul. 7, 2023.

(51) Int. Cl. A61B 5/145; A61B 5/00; A61B 5/1477; B33Y 80/00; C12N 9/04; C12N 9/08

(52) U.S. Cl. CPC A61B 5/14517; A61B 5/1477; A61B 5/6833; C12N 9/0006; C12N 9/0065; A61B 2560/0412; B33Y 80/00; C12Y 101/03; C12Y 111/01007

(57) ABSTRACT

Disclosed herein is a printable liquid lactate sensor composition including silk fibroin in an amount by weight of between 0.1% and 30%, a lactate oxidase that is activated by lactate to produce hydrogen peroxide, a peroxidase that is activated by the hydrogen peroxide, and a chromogenic substrate that changes color upon activation of the peroxidase. Wearable sensors including the printable liquid lactate sensor composition are disclosed herein.

### CLAIM TO PRIORITY

This application claims benefit of and is a continuation of International Patent Application No. PCT/US2024/014999 (Attorney Docket. No. 2095.0592), filed Feb. 8, 2024, and entitled “PAPER-BASED WEARABLE PATCHES FOR REAL-TIME, QUANTITATIVE LACTATE MONITORING,” International Pub. No. WO2024168148, which is hereby incorporated by reference in its entirety for all purposes.

International Patent Application No. PCT/US2024/014999 relates to, incorporates by reference for all purposes, and claims priority to U.S. Application Ser. No. 63/483,959 (Attorney Docket. No. 2095.0488), filed Feb. 8, 2023, and U.S. Application Ser. No. 63/512,534 (Attorney Docket. No. 2095.0554), filed Jul. 7, 2023.

### STATEMENT REGARDING FEDERALLY FUNDED RESEARCH

This invention was made with government support under Grant number N00014-19-1-2399 awarded by the US Navy, Office of Naval Research. The government has certain rights in the invention.

### SEQUENCE LISTING

Not applicable.

### BACKGROUND

Wearable sensors can be used for real-time continuous health monitoring in health care and wellness. In particular, the use of flexible interfaces that conform to the skin have attracted considerable interest for the extraction of meaningful pathophysiological information through continuous and painless sampling and analysis of biofluids. In contrast, conventional techniques for biomarkers analysis are difficult to adapt to real-time portable monitoring due to their invasive sampling protocols, biosample preparation and reagent stabilization. What is needed is a shelf-stable, non-invasive, wearable sensor.

### SUMMARY

Disclosed herein are silk-based colorimetric wearable sensing patches for lactate concentration and pH monitoring in sweat. These sensing patches can be comfortably worn during exercise or day-to-day activities and address the drawbacks of existing wearable sensing technologies by providing long shelf-life, wide sensing range, high reproducibility, and compactness. The use of silk-fibroin as a stabilizer of biological labile components is broadened, proving its applicability in the fabrication of shelf-stable sensing patches which can be used after years of storage. These flexible silk-based sensors open the way toward real-time detection of multiple analytes for continuous health and performance monitoring. Finally, pairing these wearable sensors with machine learning models for image recognition provides rapid and quantitative readouts characterized by high accuracy, high sensitivity, and sensing range suitable both for applications in sport medicine and clinical settings.

In some aspects, the techniques described herein relate to a printable liquid lactate sensor composition, the composition including: silk fibroin in an amount by weight of between 0.1% and 30%; a lactate oxidase that is activated by lactate to produce hydrogen peroxide; a peroxidase that is activated by the hydrogen peroxide; and a chromogenic substrate that changes color upon activation of the peroxidase.

In some aspects, the techniques described herein relate to a wearable sensor for detecting lactate, the wearable sensor including: a biopolymer substrate having embedded therein a lactate oxidase that is activated by the lactate to produce hydrogen peroxide, a peroxidase that is activated by the hydrogen peroxide, and a chromogenic substrate that changes color upon activation of the peroxidase, the biopolymer substrate including silk fibroin in an amount by weight of between 1% and 100%, wherein a quantitative bulk colorimetric change of the biopolymer substrate varies as a lactate concentration within the biopolymer substrate varies from 0.1 mM to 100 mM, including but not limited to, from 1 mM to 90 mM, from 0.5 mM to 70 mM, from 10 mM to 50 mM, including but not limited to a range with a lower limit or 0.1 mM, 0.5 mM, 1 mM, or 10 mM and an upper limit of 100 mM, 90 mM, 70 mM, or 50 mM.

In some aspects, the techniques described herein relate to a sweat sensor including: a biopolymer substrate having embedded therein a lactate oxidase that is activated by the lactate to produce hydrogen peroxide, a peroxidase that is activated by the hydrogen peroxide, and a chromogenic substrate that changed color upon activation of the peroxidase, the biopolymer substrate including silk fibroin in an amount by weight of between 1% and 100%, wherein a quantitative bulk colorimetric change of the biopolymer substrate varies as a lactate concentration within the biopolymer substrate varies from 0.1 mM to 100 mM, including but not limited to, from 1 mM to 90 mM, from 0.5 mM to 70 mM, from 10 mM to 50 mM, including but not limited to a range with a lower limit or 0.1 mM, 0.5 mM, 1 mM, or 10 mM and an upper limit of 100 mM, 90 mM, 70 mM, or 50 mM.

In some aspects, the techniques described herein relate to a colorimetric sensor for detecting a target chemical in a fluid sample, the sensor including: one or more detection regions on a substrate, wherein the detection regions include silk fibroin, one or more enzymatic reagents configured to detect one or more target chemicals in the fluid sample; and one or more chromogenic substrates configured to indicate a relative amount of one or more target chemicals in the fluid sample.

In some aspects, the techniques described herein relate to a method for detecting a target chemical in a fluid sample, the method including: training a target chemical detection model using a plurality of images of colorimetric sensors, wherein the sensors include a plurality of reference detection regions, and a plurality of sample detection regions having predetermined concentrations of a target chemical in a fluid sample; and predicting, using the trained target chemical detection model, a concentration of the target chemical on a colorimetric sensor.

In some aspects, the techniques described herein relate to a method of fabricating a colorimetric sensor for detecting a target chemical in a sample fluid, the method including: preparing one or more paper substrates with at least one of a silk fibroin solution, one or more enzymatic reagents, or one or more chromogenic substrates; and placing the one or more paper substrates on a film substrate.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIG. 1 depicts: A) Fabrication schematic of the paper-based lactate-sensing patches. Lactate oxidase (LOx), horseradish peroxidase (HRP) and dyes are added to a silk fibroin solution to form a chromogenic enzymatic ink, which is then drop-cast on paper. Circular lactate-sensing interfaces are laser-cut and applied on Tegaderm™ films to obtain wearable patches whose color shifts from yellow to dark red by increasing the concentration of lactate in sweat. B) Schematic representation of the LOx/HRP cascade reaction. LOx oxidizes lactate to produce pyruvate and hydrogen peroxide, which is used by HRP to oxidize the chromogenic substrates generating a visible color change. C) Photographs of the lactate-sensing patches applied on skin. The insets show the circular lactate-sensing interfaces before (top) and after (bottom) the colorimetric response.

FIG. 2 depicts: A) Calibration curve for the silk-based chromogenic enzymatic ink with and without the deposition of a base layer of chitosan on Whatman grade 1 filter paper. Sensing range with chitosan: 0-90 mM; sensing range without chitosan: 0-50 mM. Colored circles show the colorimetric response recorded at different lactate concentrations. B) Sensing range and sensitivity values after 8, 24 and 120 h of storage at 60° C. for the silk-based chromogenic enzymatic ink with and without the deposition of a base layer of chitosan on Whatman grade 1 filter paper. Inset shows the colorimetric response of the silk-based chromogenic enzymatic ink with a base layer of chitosan on Whatman grade 1 filter paper after 120 h of storage at 60° C. C) Calibration curve for the silk-based and water-based chromogenic enzymatic inks with a base layer of chitosan on Whatman grade 1 filter paper. Sensing range of silk-based ink: 0-90 mM; sensing range of water-based ink: 0-10 mM. Colored circles show the colorimetric response recorded at different lactate concentrations. D) Sensing range and sensitivity values after 8, 24 and 120 h of storage at 60° C. for the silk-based and water-based chromogenic enzymatic inks with a base layer of chitosan on Whatman grade 1 filter paper. Inset shows the colorimetric response of the water-based chromogenic enzymatic ink with a base layer of chitosan on Whatman grade 1 filter paper after 120 h of storage at 60° C. E) Retained activity after 18, 21 and 24 months of storage at 4° C. for the silk-based and water-based chromogenic enzymatic inks with a base layer of chitosan on Whatman grade 1 filter paper. Insets show the colorimetric response of the silk-based and water-based chromogenic enzymatic ink 24 months of storage at 4° C. F) Retained activity after 8 h, 24 h, 5 days and 2.5 months of storage at 60° C. for the silk-based and water-based chromogenic enzymatic inks with a base layer of chitosan on Whatman grade 1 filter paper. Insets show the colorimetric response of the silk-based and water-based chromogenic enzymatic ink 2.5 months of storage at 60° C.

FIG. 3 depicts: A) Calibration curve for bromocresol green (BG) silk-based chromogenic pH-sensing inks on Whatman grade 1 filter paper. Sensitivity: −39.8±1.3. Sensing range: pH 3-7. B) Calibration curve for the nitrazine yellow (NY) silk-based chromogenic pH-sensing inks on Whatman grade 1 filter paper. Sensitivity: −76.1±1.4. Sensing range: pH 5.5-7.5. C) Calibration curve for the phenol red (PR) silk-based chromogenic pH-sensing inks on Whatman grade 1 filter paper. Sensitivity: −40.9±1.9. Sensing range: pH 6.5-8.5. Colored circles show the colorimetric response recorded at different pH for each ink. D) Sensing range of bromocresol green, nitrazine yellow and phenol red silk-based chromogenic pH-sensing inks on Whatman grade 1 filter paper.

FIG. 4 depicts: A) Schematic of the SVM model training and lactate prediction process. Sweat with a known lactate concentration is drop-cast on the sensor. After the colorimetric response, images of the sensor are acquired in different light conditions. The images are used to train the SVM model and to evaluate its performances. The trained SVM model is able to predict the lactate concentration when an image of the sensor is given as input. B) (Top) Scatterplot of the full dataset in the three-dimensional RGB color space for the different lactate concentrations (0-50 mM). (Bottom) Scatterplot of the full dataset in the two-dimensional Blue Channel vs. Red Channel plane for the different lactate concentrations (0-50 mM). C) The SVM model was trained using 1316 images of the colorimetric response of the wearable sensor upon exposition to known lactate concentrations in the 0-50 mM range. D) (Top) The performances of the SVM model were evaluated using 564 images of the colorimetric response of the wearable sensor upon exposition to known lactate concentrations in the 0-50 mM range. (Bottom) Confusion matrix for the 564 evaluation images showing that the SVM model accurately classified the images into the 6 lactate concentration categories with an overall accuracy of 89.3%. E) Images of the colorimetric response of the wearable sensors before (left), during (center) and after (right) a treadmill exercise session. The predicted lactate concentration at the end of the session was 30 mM.

FIG. 5 depicts the sensor's colorimetric change in the Green Channel before and after the exposure to interfering substances (NaCl, KCl, Urea, NH₄Cl, CaCl₂), MgCl₂, with concentrations of 40 mM, 3 mM, 22 mM, 3 mM, 0.4 mM, 50 M respectively) compared to the colorimetric change when exposed to lactate with a concentration of 5 mM.

FIG. 6 depicts: A) Calibration curve for the silk-based chromogenic enzymatic ink with and without the deposition of a base layer of chitosan on Ahlstrom grade 55 filter paper. B) Sensing range and sensitivity values after 8, 24 and 120 h of storage at 60° C. for the silk-based chromogenic enzymatic ink with and without the deposition of a base layer of chitosan on Ahlstrom grade 55 filter paper. C) Calibration curve for the silk-based and water-based chromogenic enzymatic inks with a base layer of chitosan on Ahlstrom grade 55 filter paper. D) Sensing range and sensitivity values after 8, 24 and 120 h of storage at 60° C. for the silk-based and water-based chromogenic enzymatic inks with a base layer of chitosan on Ahlstrom grade 55 filter paper.

FIG. 7 depicts: A) Calibration curve for the silk-based chromogenic enzymatic ink with and without the deposition of a base layer of chitosan on Whatman grade 4 filter paper. B) Sensing range and sensitivity values after 8, 24 and 120 h of storage at 60° C. for the silk-based chromogenic enzymatic ink with and without the deposition of a base layer of chitosan on Whatman grade 4 filter paper. C) Calibration curve for the silk-based and water-based chromogenic enzymatic inks with a base layer of chitosan on Whatman grade 4 filter paper. D) Sensing range and sensitivity values after 8, 24 and 120 h of storage at 60° C. for the silk-based and water-based chromogenic enzymatic inks with a base layer of chitosan on Whatman grade 4 filter paper.

### DETAILED DESCRIPTION

Before the present disclosure is described in further detail, it is to be understood that the disclosure is not limited to the particular embodiments described. It is also understood that the terminology used herein is for the purpose of describing particular embodiments only, and is not intended to be limiting. The scope of the present disclosure will be limited only by the claims. As used herein, the singular forms “a”, “an”, and “the” include plural embodiments unless the context clearly dictates otherwise.

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; and (v) where ranges are provided, endpoints are included.

Approximately: as used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Composition: as used herein, may be used to refer to a discrete physical entity that comprises one or more specified components. In general, unless otherwise specified, a composition may be of any form—e.g., gas, gel, liquid, solid, etc. In some embodiments, “composition” may refer to a combination of two or more entities for use in a single embodiment or as part of the same article. It is not required in all embodiments that the combination of entities result in physical admixture, that is, combination as separate co-entities of each of the components of the composition is possible; however many practitioners in the field may find it advantageous to prepare a composition that is an admixture of two or more of the ingredients in a pharmaceutically acceptable carrier, diluent, or excipient, making it possible to administer the component ingredients of the combination at the same time.

Improve, increase, or reduce: as used herein or grammatical equivalents thereof, indicate values that are relative to a baseline measurement, such as a measurement in a similar composition made according to previously known methods.

Substantially: as used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “silk fibroin” refers to silk fibroin protein whether produced by silkworm, spider, or other insect, or otherwise generated (Lucas et al., Adv. Protein Chem., 13: 107-242 (1958)). Any type of silk fibroin can be used in different embodiments described herein. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein by reference in their entireties.

Disclosed herein is a shelf-stable, non-invasive, paper-based colorimetric wearable lactate sensor. This sensor exploits the ability of silk to control the concentration, print, and functionally preserve labile transducing biomolecules in the format of a shelf-stable digital patch for optical readout. This novel approach overcomes major challenges associated with the commercialization of colorimetric wearable sensors (e.g., enzyme thermal instability, narrow sensing range, low sensitivity, and qualitative response) by showing a combination of unprecedented stability (i.e., up to 2 years in refrigerated conditions), wide sensing range, and high sensitivity. Additionally, real-time quantitative signal readouts are achieved using machine learning-driven image analysis enabling physiological status evaluation with a simple smartphone camera.

Non-invasive continuous health monitoring enables access to physiological and pathological information to assess human wellbeing. In this context, wearable sensors provide great utility providing painless sampling of biofluids and real-time analysis of relevant biomarkers, with emerging applications in healthcare and sport medicine. Both electrochemical and colorimetric enzymatic wearable sensors have been widely studied for this purpose, yet challenges are still present in this important application space where the electrochemical sensors can be bulky and not comfortable to wear while colorimetric sensors exhibit narrow sensing range and provide binary or qualitative responses, which are hard to read by eye. The commercialization of both types of sensors faces limitations imposed by the biological transduction mechanism, where instability of the enzymes used for detection significantly decreases the sensor shelf-life.

Disclosed herein is a shelf-stable, skin mounted, non-invasive paper-based colorimetric wearable lactate sensor that combines high sensitivity and wide linear sensing range by employing composite inks formed by a silk fibroin and chromogenic enzymatic mixture. The composite ink exploits the demonstrated ability of silk fibroin to stabilize labile entities (e.g., enzymes, cells, small molecules, proteins, nucleic acids, antioxidants, perishable products) to generate sweat sensing patches with long shelf-life (i.e., 2 years under refrigerated conditions). These patches are combined with machine learning-assisted image analysis to provide quantitative real-time readings with high sensitivity (i.e., 80-100%) and specificity (i.e., 95-100%).

Sweat is of particular interest as it is relatively easy to access and contains several biomarkers correlated to physical stress, dehydration, infections, and diseases. Sweat monitoring can reveal important information regarding patients' and athletes' physiological status. The distribution of sweat glands across the skin enables its sampling over the entire body making it an excellent biofluid both for localized and distributed sensing. In counterpoint to its sampling ease, sweat is a complex biofluid affected by environmental and physiological interferences that make its analysis challenging.

As the end product of glycolysis, lactate in sweat is a target of interest for non-invasive monitoring. Lactate levels provide insights about the health status of patients and is used as a diagnostic biomarker for oxygen deficiency conditions caused by compromised oxygen transport, as well as being a predictor of mortality in trauma patients. Additionally, its production increases during high-intensity physical activity in relation to the level of fatigue faced during exercise and to the fitness level of the subject. While its accumulation causes soreness that can deter from further physical activity, its production is deemed essential to improve endurance. Consequently, lactate monitoring can improve athletes' performances while also preventing injuries caused by overtraining. To date, conventional lactate detection techniques rely on blood samples and are not suitable for real-time portable monitoring due to their invasive sampling protocols (e.g., venipuncture or finger prick). The wearable sensors disclosed herein are light, flexible, conformable to the skin and can be worn on different parts of the body for long periods of time providing continuous distributed sampling of sweat without causing discomfort. The sensors disclosed herein may be obtained by functionalizing filter paper by applying one or more layers of sensing inks (e.g., 1, 2, 3, etc.), such as by drop-casting or ink-jet printing. In embodiments, drop-casting requires no thickening agent as the inks are liquid solutions of relatively low viscosity. In embodiments, sensors disclosed herein may include filter paper. Without wishing to be bound by any particular theory, the smaller pore sizes of some filter papers may be why they exhibit the best results in terms of sensing range.

Disclosed herein is a printable liquid lactate sensor composition including silk fibroin in an amount by weight of between 0.1% and 30%, a lactate oxidase that is activated by lactate to produce hydrogen peroxide, a peroxidase that is activated by the hydrogen peroxide, and a chromogenic substrate that changes color upon activation of the peroxidase.

Also disclosed herein is a solid sensor including a biopolymer substrate formed from the printable liquid lactate sensor composition. The lactate oxidase, the peroxidase, and the chromogenic substrate may be embedded in the biopolymer substrate.

Also disclosed herein is a wearable sensor for detecting lactate including a biopolymer substrate that has embedded lactate oxidase that is activated by the lactate to produce hydrogen peroxide, a peroxidase that is activated by the hydrogen peroxide, and a chromogenic substrate that changes color upon activation of the peroxidase. The biopolymer substrate includes silk fibroin in an amount by weight of between 1% and 100%.

Also disclosed herein is a sweat sensor including a biopolymer substrate having embedded therein a lactate oxidase that is activated by the lactate to produce hydrogen peroxide, a peroxidase that is activated by the hydrogen peroxide, and a chromogenic substrate that changes color upon activation of the peroxidase. The biopolymer substrate includes silk fibroin in an amount by weight of between 1% and 100%.

The sensing range of the sensors disclosed herein (e.g., the solid sensor, the wearable sensor, the sweat sensor, lactate sensor, etc.) significantly exceeds other known lactate sensing systems, which in some embodiments may be a more than three-fold increase in the concentration of lactate that can be sensed. The inventors surprisingly achieved a quantitative bulk colorimetric change of the biopolymer substrate that varies as a lactate concentration within the biopolymer substrate varies from 0.1 mM to 100 mM. In some embodiments, the quantitative bulk colorimetric change was achieved at lactate concentrations varying from 1 mM to 90 mM. In some embodiments, the quantitative bulk colorimetric change was achieved at lactate concentrations varying from 0.5 mM to 70 mM. In some embodiments, the quantitative bulk colorimetric change was achieved at lactate concentrations varying from 10 mM to 50 mM. In some embodiments, the quantitative bulk colorimetric change was achieved at lactate concentrations in a range with a lower limit of 0.1 mM, 0.5 mM, 1 mM, or 10 mM. In some embodiments, the quantitative bulk colorimetric change was achieved at lactate concentrations in a range with an upper limit of 100 mM, 90 mM, 70 mM, or 50 mM. In embodiments, the bulk quantitative colorimetric change occurs substantially immediately upon contact with a solution having a different lactate concentration.

In embodiments of the compositions or sensors disclosed herein, the chromogenic substrate of the composition and/or the biopolymer substrate includes a baseline colorant, such as a yellow dye (e.g., an acid yellow dye such as Acid Yellow 34). The chromogenic substrate can also include sodium 3,5-dichloro-2-hydroxygenzenesulfonate and/or 4-aminoantipyrine. In some cases, the chromogenic substrate includes a yellow dye, such as an acid yellow dye, such as Acid Yellow 34, sodium 3,5-dichloro-2-hydroxygenzenesulfonate and 4-aminoantipyrine. In embodiments of the compositions or sensors disclosed herein, the composition and/or the biopolymer substrate include a baseline buffer and/or electrolyte mixture, wherein the baseline buffer and/or electrolyte mixture is optionally tailored to mimic human sweat. In embodiments of the sensors disclosed herein, the biopolymer substrate may include a base layer of chitosan.

Disclosed herein are articles of clothing or wearable patches including a plurality of the sensors disclosed herein. The articles of clothing or wearable patches may include reference color spots having predetermined known colors for colorimetric analysis of images of the sensor or sensors.

Disclosed herein is a colorimetric sensor for detecting a target chemical in a fluid sample including one or more detection regions on a substrate, one or more enzymatic reagents configured to detect one or more target chemicals in the fluid sample, and one or more chromogenic substrates configured to indicate a relative amount of one or more target chemicals in the fluid sample. In embodiments, the detection regions include silk fibroin. In embodiments, the detection regions include chitosan. In embodiments, the colorimetric sensor may include an imaging device (e.g., a multi-spectral camera) for detecting a colorimetric change in the one or more detector regions after contact with the fluid sample including the one or more target chemical for the one or more enzymatic reagents. A processor may be connected to the imaging device and configured to detect the target enzyme and quantify the amount of the target enzyme in the fluid. The processor may include a machine-learning model configured to train the sensor to detect and quantify a chemical in a fluid sample. The machine-learning model may be trained using a plurality of images of colorimetric sensor responsive to known concentrations of the one or more target chemicals. In embodiments, the one or more enzymatic reagents includes lactate oxidase (LOx) and may further include horse radish peroxidase (HRP). The target chemical may include lactate. In embodiments, the fluid sample includes a biological fluid (e.g., sweat). In embodiments, the substrate includes a flexible material configured to overlay and conform to a sensing surface. In embodiments, the colorimetric sensor further includes one or more pH sensing regions on the substrate configured to detect a pH level of the fluid sample. The pH sensing regions may include one or more chromogenic pH sensing indicators which may define a pH range. In embodiments, a plurality of the detection regions may be arranged in a predetermined pattern. The processor may generate a spatial distribution map of the one or more target chemical based on the predetermined pattern of the detection regions.

Disclosed herein is a method for detecting a target chemical in a fluid sample including training a target chemical detection model using a plurality of images of colorimetric sensors, wherein the sensors include a plurality of reference detection regions and a plurality of sample detection regions having predetermined concentrations of a target chemical in a fluid sample, and predicting, using the trained target chemical detection model, a concentration of the target chemical on a colorimetric sensor. In embodiments, the plurality of images may be acquired in a plurality of light conditions, or may be acquired using a multispectral camera. In embodiments, the plurality of images is acquired using an imaging device for detecting a colorimetric change in the plurality of sample detection regions after contact with the fluid sample including the target chemical. In embodiments, the plurality of images may be divided into a plurality of categories of the predetermined concentrations of the target chemical. In embodiments, the plurality of images are labeled with their respective concentrations of the target chemical, and combined into a dataset.

Disclosed herein is a method of fabricating a colorimetric sensor for detecting a target chemical in a sample fluid including preparing one or more paper substrates with at least one of a silk fibroin solution, one or more enzymatic reagents (e.g., lactate oxidase (LOx)), or one or more chromogenic substrates, and placing the one or more paper substrates on a film substrate. The method may further include preconditioning the one or more paper substrates with a chitosan solution. The one or more enzymatic reagents may further include horse radish peroxidase (HRP). In embodiments, the method may further include placing one or more pH sensing regions on the film substrate configured to detect a pH level of the sample fluid. The pH sensing regions may include one or more chromogenic pH sensing indicators which may define a pH range. In embodiments, the one or more paper substrates may be arranged in a predetermined pattern on the film substrate.

According to various embodiments, a variety of functionalizing agents may be used with the sensors and other embodiments described herein.

According to various embodiments, any application-appropriate amount of one or more functionalizing agents may be used. In some embodiments, the amount of an individual functionalizing agent may be between about 1 g/ml and 1,000 g/ml (e.g., between about 2 and 1,000, 5 and 1,000, 10 and 1,000, 10 and 500, 10 and 100 g/ml). In some embodiments, the amount of an individual functionalizing agent may be at least 1 g/ml (e.g., at least 5, 10, 15, 20 25, 50, 100, 200, 300 400, 500, 600, 700, 800, or 900 g/ml). In some embodiments, the amount of an individual functionalizing agent is at most 1,000 g/ml (e.g., 900, 800, 700, 600, 500, 400, 300 200, 100, 90, 80, 70, 60, 50, 40, 30, 20, 10, or 5 g/ml).

In some aspects, the functionalizing agent may include one or more sensing agents, such as a sensing dye. The sensing agents/sensing dyes are environmentally sensitive and produce a measurable response to one or more environmental factors. In some aspects, the environmentally-sensitive agent or dye may be present in the composition in an effective amount to alter the composition from a first chemical-physical state to a second chemical-physical state in response to an environmental parameter (e.g., a change in pH, light intensity or exposure, temperature, pressure or strain, voltage, physiological parameter of a subject, and/or concentration of chemical species in the surrounding environment) or an externally applied stimulus (e.g., optical interrogation, acoustic interrogation, and/or applied heat). In some cases, the sensing dye is present to provide one optical appearance under one given set of environmental conditions and a second, different optical appearance under a different given set of environmental conditions. Suitable concentrations for the sensing agents described herein can be the concentrations for the colorants and additives described elsewhere herein. A person having ordinary skill in the chemical sensing arts can determine a concentration that is appropriate for use in a sensing application of the inks described herein.

In some aspects, the first and second chemical-physical state may be a physical property of the composition, such as mechanical property, a chemical property, an acoustical property, an electrical property, a magnetic property, an optical property, a thermal property, a radiological property, or an organoleptic property. Exemplary sensing dyes or agents include, but are not limited to, a pH sensitive agent, a thermal sensitive agent, a pressure or strain sensitive agent, a light sensitive agent, or a potentiometric agent.

Exemplary pH sensitive dyes or agents include, but are not limited to, cresol red, methyl violet, crystal violet, ethyl violet, malachite green, methyl green, 2-(p-dimethylaminophenylazo) pyridine, paramethyl red, metanil yellow, 4-phenylazodiphenylamine, thymol blue, metacresol purple, orange IV, 4-o-Tolylazo-o-toluindine, quinaldine red, 2,4-dinitrophenol, erythrosine disodium salt, benzopurpurine 4B, N,N-dimethyl-p-(m-tolylazo) aniline, p-dimethylaminoazobenene, 4,4′-bis(2-amino-1-naphthylazo)-2,2′-stilbenedisulfonic acid, tetrabromophenolphthalein ethyl ester, bromophenol blue, Congo red, methyl orange, ethyl orange, 4-(4-dimethylamino-1-naphylazo)-3-methoxybenesulfonic acid, bromocresol green, resazurin, 4-phenylazo-1-napthylamine, ethyl red 2-(1-dimethylaminophenyazo) pyridine, 4-(p-ethoxypehnylazo)-m-phenylene-diamine monohydrochloride, resorcin blue, alizarin red S, methyl red, propyl red, bromocresol purple, chlorophenol red, p-nitrophenol, alizarin 2-(2,4-dinitrophenylazo) 1-napthol-3,6-disulfonic acid, bromothymol blue, 6,8-dinitro-2,4-(1H) quinazolinedione, brilliant yellow, phenol red, neutral red, m-nitrophenol, cresol red, turmeric, metacresol purple, 4,4′-bis(3-amino-1-naphthylazo)-2,2′-stilbenedisulfonic acid, thymol blue, p-naphtholbenzein, phenolphthalein, o-cresolphthalein, ethyl bis(2,4-dimethylphenyl) ethanoate, thymolphthalein, nitrazine yellow, alizarin yellow R, alizarin, p-(2,4-dihydroxyphenylazo) benzenesulfonic acid, 5,5′-indigodisulfonic acid, 2,4,6-trinitrotoluene, 1,3,5-trinitrobenzene, and clayton yellow.

Exemplary light responsive dyes or agents include, but are not limited to, photochromic compounds or agents, such as triarylmethanes, stilbenes, azasilbenes, nitrones, fulgides, spiropyrans, napthopyrans, spiro-oxzines, quinones, derivatives and combinations thereof.

Exemplary potentiometric dyes include, but are not limited to, substituted amiononaphthylehenylpridinium (ANEP) dyes, such as di-4-ANEPPS, di-8-ANEPPS, and N-(4-Sulfobutyl)-4-(6-(4-(Dibutylamino)phenyl)hexatrienyl)Pyridinium (RH237).

Exemplary temperature sensitive dyes or agents include, but are not limited to, thermochromic compounds or agents, such as thermochromic liquid crystals, leuco dyes, fluoran dyes, octadecylphosphonic acid.

Exemplary pressure or strain sensitive dyes or agents include, but are not limited to, spiropyran compounds and agents.

Exemplary chemi-sensitive dyes or agents include, but are not limited to, antibodies such as immunoglobulin G (IgG) which may change color from blue to red in response to bacterial contamination.

In some aspects, the functionalizing agent comprises one or more additive, dopant, or biologically active agent suitable for a desired intended purpose. In some aspects, the additive or dopant may be present in an amount effective to impart an optical or organoleptic property to the composition. Exemplary additives or dopants that impart optical or organoleptic properties include, but are not limited to, dyes/pigments, flavorants, aroma compounds, granular or fibrous fillers.

Additionally or alternatively, the additive, dopant, or biologically active agent may be present in an amount effective to “functionalize” the composition to impart a desired mechanical property or added functionality to the composition. Exemplary additive, dopants, or biologically active agent that impart the desired mechanical property or added functionality include, but are not limited to: environmentally sensitive/sensing dyes; active biomolecules; conductive or metallic particles; micro and nanofibers (e.g., silk nanofibers for reinforcement, carbon nanofibers); nanotubes; inorganic particles (e.g., hydroxyapatite, tricalcium phosphate, bioglasses); drugs (e.g., antibiotics, small molecules or low molecular weight organic compounds); proteins and fragments or complexes thereof (e.g., enzymes, antigens, antibodies and antigen-binding fragments thereof); DNA/RNA (e.g., siRNA, miRNA, mRNA); cells and fractions thereof (viruses and viral particles; prokaryotic cells such as bacteria; eukaryotic cells such as mammalian cells and plant cells; fungi).

In some aspects, the additive or dopant comprises an aroma compound. Exemplary aroma compounds include ester aroma compounds, terpene aroma compounds, cyclic terpenes, and aromatic aroma compounds, such as, but not limited to, geranyl acetate, methyl formate, methyl acetate, methyl propionate, methyl butyrate, ethyl acetate, ethyl butyrate, isoamyl acetate, pentyl butrate, pentyl pentanoate, octyl acetate, benzyl acetate, methyl anthranilate, myrecene, geraniol, nerol, citral, cironellal, cironellol, linalool, nerolidol, limonene, camphor, menthol, carone, terpineol, alpha-lonone, thujone, eucalyptol, benzaldehyde, eugenol, cinnamaldehyde, ethyl maltol, vanillin, anisole, anethole, estragole, thymol.

In some aspects, the additive or dopant comprises a colorant, such as a dye or pigment. In some aspects, the dye or pigment imparts a color or grayscale to the composition. The colorant can be different than the sensing agents and/or sensing dyes below. Any organic and/or inorganic pigments and dyes can be included in the inks. Exemplary pigments suitable for use in the present disclosure include International Color Index or C.I. Pigment Black Numbers 1, 7, 1 1 and 31, C.I. Pigment Blue Numbers 15, 15:1, 15:2, 15:3, 15:4, 15:6, 16, 27, 29, 61 and 62, C.I. Pigment Green Numbers 7, 17, 18 and 36, C.I. Pigment Orange Numbers 5, 13, 16, 34 and 36, C.I. Pigment Violet Numbers 3, 19, 23 and 27, C.I. Pigment Red Numbers 3, 17, 22, 23, 48:1, 48:2, 57:1, 81:1, 81:2, 81:3, 81:5, 101, 1 14, 122, 144, 146, 170, 176, 179, 181, 185, 188, 202, 206, 207, 210 and 249, C.I. Pigment Yellow Numbers 1, 2, 3, 12, 13, 14, 17, 42, 65, 73, 74, 75, 83, 30, 93, 109, 1 10, 128, 138, 139, 147, 142, 151, 154 and 180, D&C Red No. 7, D&C Red No. 6 and D&C Red No. 34, carbon black pigment (such as Regal 330, Cabot Corporation), quinacridone pigments (Quinacridone Magenta (228-0122), available from Sun Chemical Corporation, Fort Lee, N.J.), diarylide yellow pigment (such as AAOT Yellow (274-1788) available from Sun Chemical Corporation); and phthalocyanine blue pigment (such as Blue 15:3 (294-1298) available from Sun Chemical Corporation). The classes of dyes suitable for use in present invention can be selected from acid dyes, natural dyes, direct dyes (either cationic or anionic), basic dyes, and reactive dyes. The acid dyes, also regarded as anionic dyes, are soluble in water and mainly insoluble in organic solvents and are selected, from yellow acid dyes, orange acid dyes, red acid dyes, violet acid dyes, blue acid dyes, green acid dyes, and black acid dyes. European Patent 0745651, incorporated herein by reference, describes a number of acid dyes that are suitable for use in the present disclosure. Exemplary yellow acid dyes include Acid Yellow 1 International Color Index or C.I. 10316); Acid Yellow 7 (C.I. 56295); Acid Yellow 17 (C.I. 18965); Acid Yellow 23 (C.I. 19140); Acid Yellow 29 (C.I. 18900); Acid Yellow 36 (C.I. 13065); Acid Yellow 42 (C.I. 22910); Acid Yellow 73 (C.I. 45350); Acid Yellow 99 (C.I. 13908); Acid Yellow 194; and Food Yellow 3 (C.I. 15985). Exemplary orange acid dyes include Acid Orange 1 (C.I. 13090/1); Acid Orange 10 (C.I. 16230); Acid Orange 20 (C.I. 14603); Acid Orange 76 (C.I. 18870); Acid Orange 142; Food Orange 2 (C.I. 15980); and Orange B.

Exemplary red acid dyes include Acid Red 1. (C.I. 18050); Acid Red 4 (C.I. 14710); Acid Red 18 (C.I. 16255), Acid Red 26 (C.I. 16150); Acid Red 2.7 (C.I. as Acid Red 51 (C.I. 45430, available from BASF Corporation, Mt. Olive, N.J.) Acid Red 52 (C.I. 45100); Acid Red 73 (C.I. 27290); Acid Red 87 (C. I. 45380); Acid Red 94 (C.I. 45440) Acid Red 194; and Food Red 1 (C.I. 14700). Exemplary violet acid dyes include Acid Violet 7 (C.I. 18055); and Acid Violet 49 (C.I. 42640). Exemplary blue acid dyes include Acid Blue 1 (C.I. 42045); Acid Blue 9 (C.I. 42090); Acid Blue 22 (C.I. 42755); Acid Blue 74 (C.I. 73015); Acid Blue 93 (C.I. 42780); and Acid Blue 158A (C.I. 15050). Exemplary green acid dyes include Acid Green 1 (C.I. 10028); Acid Green 3 (C.I. 42085); Acid Green 5 (C.I. 42095); Acid Green 26 (C.I. 44025); and Food Green 3 (C.I. 42053). Exemplary black acid dyes include Acid Black 1 (C.I. 20470); Acid Black 194 (Basantol® X80, available from BASF Corporation, an azo/1:2 CR-complex.

Exemplary direct dyes for use in the present disclosure include Direct Blue 86 (C.I. 74180); Direct Blue 199; Direct Black 168; Direct Red 253; and Direct Yellow 107/132 (C.I. Not Assigned).

Exemplary natural dyes for use in the present disclosure include Alkanet (C.I. 75520,75530); Annafto (C.I. 75120); Carotene (C.I. 75130); Chestnut; Cochineal (C.I. 75470); Cutch (C.I. 75250, 75260); Divi-Divi; Fustic (C.I. 75240); Hypernic (C.I. 75280); Logwood (C.I. 75200); Osage Orange (C.I. 75660); Paprika; Quercitron (C.I. 75720); Sanrou (C.I. 75100); Sandal Wood (C.I. 75510, 75540, 75550, 75560); Sumac; and Tumeric (C.I. 75300). Exemplary reactive dyes for use in the present disclosure include Reactive Yellow 37 (monoazo dye); Reactive Black 31 (disazo dye); Reactive Blue 77 (phthalo cyanine dye) and Reactive Red 180 and Reactive Red 108 dyes. Suitable also are the colorants described in The Printing Ink Manual (5th ed., Leach et al. eds. (2007), pages 289-299. Other organic and inorganic pigments and dyes and combinations thereof can be used to achieve the colors desired.

In addition to or in place of visible colorants, compositions provided herein can contain ETV fluorophores that are excited in the ETV range and emit light at a higher wavelength (typically 400 nm and above). Examples of ETV fluorophores include but are not limited to materials from the coumarin, benzoxazole, rhodamine, napthalimide, perylene, benzanthrones, benzoxanthones or benzothia-xanthones families. The addition of a UV fluorophore (such as an optical brightener for instance) can help maintain maximum visible light transmission. The amount of colorant, when present, generally is between 0.05% to 5% or between 0.1% and 1% based on the weight of the composition.

For non-white compositions, the amount of pigment/dye generally is present in an amount of from at or about 0.1 wt % to at or about 20 wt % based on the weight of the composition. In some applications, a non-white ink can include 15 wt % or less pigment/dye, or 10 wt % or less pigment/dye or 5 wt % pigment/dye, or 1 wt % pigment/dye based on the weight of the composition. In some applications, a non-white ink can include 1 wt % to 10 wt %, or 5 wt % to 15 wt %, or 10 wt % to 20 wt % pigment/dye based on the weight of the composition. In some applications, a non-white ink can contain an amount of dye/pigment that is 1 wt %, 2 wt %, 3 wt %, 4 wt %, 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt % or 20 wt % based on the weight of the composition.

For white compositions, the amount of white pigment generally is present in an amount of from at or about 1 wt % to at or about 60 wt % based on the weight of the composition. In some applications, greater than 60 wt % white pigment can be present. Preferred white pigments include titanium dioxide (anatase and rutile), zinc oxide, lithopone (calcined coprecipitate of barium sulfate and zinc sulfide), zinc sulfide, blanc fixe and alumina hydrate and combinations thereof, although any of these can be combined with calcium carbonate. In some applications, a white ink can include 60 wt % or less white pigment, or 55 wt % or less white pigment, or 50 wt % white pigment, or 45 wt % white pigment, or 40 wt % white pigment, or 35 wt % white pigment, or 30 wt % white pigment, or 25 wt % white pigment, or 20 wt % white pigment, or 15 wt % white pigment, or 10 wt % white pigment, based on the weight of the composition. In some applications, a white ink can include 5 wt % to 60 wt %, or 5 wt % to 55 wt %, or 10 wt % to 50 wt %, or 10 wt % to 25 wt %, or 25 wt % to 50 wt %, or 5 wt % to 15 wt %, or 40 wt % to 60 wt % white pigment based on the weight of the composition. In some applications, a non-white ink can an amount of dye/pigment that is 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt %, 20 wt %, 21 wt %, 22 wt %, 23 wt %, 24 wt %, 25%, 26 wt %, 27 wt %, 28 wt %, 29 wt %, 30 wt %, 31 wt %, 32 wt %, 33 wt %, 34 wt %, 35%, 36 wt %, 37 wt %, 38 wt %, 39 wt %, 40 wt %, 41 wt %, 42 wt %, 43 wt %, 44 wt %, 45%, 46 wt %, 47 wt %, 48 wt %, 49 wt %, 50 wt %, 51 wt %, 52 wt %, 53 wt %, 54 wt %, 55%, 56 wt %, 57 wt %, 58 wt %, 59 wt % or 60 wt % based on the weight of the composition.

In some aspects, the additive or dopant comprises a conductive additive. Exemplary conductive additives include, but are not limited to graphite, graphite powder, carbon nanotubes, and metallic particles or nanoparticles, such as gold nanoparticles. In some aspects, the conductive additive is biocompatible and non-toxic.

In some aspects, the functionalizing agent is a wound healing agent. As used herein, a “wound healing agent” is a compound or composition that actively promotes wound healing process.

Exemplary wound healing agents include, but are not limited to dexpanthenol; growth factors; enzymes, hormones; povidon-iodide; fatty acids; anti-inflammatory agents; antibiotics; antimicrobials; antiseptics; cytokines; thrombin; angalgesics; opioids; aminoxyls; furoxans; nitrosothiols; nitrates and anthocyanins; nucleosides, such as adenosine; and nucleotides, such as adenosine diphosphate (ADP) and adenosine triphosphate (ATP); neutotransmitter/neuromodulators, such as acetylcholine and 5-hydroxytryptamine (serotonin/5-HT); histamine and catecholamines, such as adrenalin and noradrenalin; lipid molecules, such as 5 sphingosine-1-phosphate and lysophosphatidic acid; amino acids, such as arginine and lysine; peptides such as the bradykinins, substance P and calcium gene-related peptide (CGRP); nitric oxide; and any combinations thereof.

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems disclosed herein. The terms computer, computing device, processor, circuit, and/or server, as utilized herein, should be understood broadly.

Any one or more of the terms computer, computing device, processor, circuit, and/or server include a computer of any type, capable to access instructions stored in communication thereto such as upon a non-transient computer readable medium, whereupon the computer performs operations of systems or methods described herein upon executing the instructions. In certain embodiments, such instructions themselves comprise a computer, computing device, processor, circuit, and/or server. Additionally or alternatively, a computer, computing device, processor, circuit, and/or server may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware, computers, computing devices, processors, circuits, and/or servers include, without limitation, a general purpose computer, a server, an embedded computer, a mobile device, a virtual machine, and/or an emulated version of one or more of these. Example and non-limiting hardware, computers, computing devices, processors, circuits, and/or servers may be physical, logical, or virtual. A computer, computing device, processor, circuit, and/or server may be: a distributed resource included as an aspect of several devices; and/or included as an interoperable set of resources to perform described functions of the computer, computing device, processor, circuit, and/or server, such that the distributed resources function together to perform the operations of the computer, computing device, processor, circuit, and/or server. In certain embodiments, each computer, computing device, processor, circuit, and/or server may be on separate hardware, and/or one or more hardware devices may include aspects of more than one computer, computing device, processor, circuit, and/or server, for example as separately executable instructions stored on the hardware device, and/or as logically partitioned aspects of a set of executable instructions, with some aspects of the hardware device comprising a part of a first computer, computing device, processor, circuit, and/or server, and some aspects of the hardware device comprising a part of a second computer, computing device, processor, circuit, and/or server.

A computer, computing device, processor, circuit, and/or server may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices utilized for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players, and the like. These mobile devices may include, apart from other components, a storage medium such as a flash memory, buffer, RAM, ROM and one or more computing devices. The computing devices associated with mobile devices may be enabled to execute methods, program code, instructions, and/or programs stored thereon. Alternatively, the mobile devices may be configured to execute instructions in collaboration with other devices. The mobile devices may communicate with base stations interfaced with servers and configured to execute methods, program code, instructions, and/or programs. The mobile devices may communicate on a peer to peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information. Operations including interpreting, receiving, and/or determining any value parameter, input, data, and/or other information include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first operation to interpret, receive, and/or determine a data value may be performed, and when communications are restored an updated operation to interpret, receive, and/or determine the data value may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g. where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The elements described and depicted herein, including in flow charts, block diagrams, and/or operational descriptions, depict and/or describe specific example arrangements of elements for purposes of illustration. However, the depicted and/or described elements, the functions thereof, and/or arrangements of these, may be implemented on machines, such as through computer executable transitory and/or non-transitory media having a processor capable of executing program instructions stored thereon, and/or as logical circuits or hardware arrangements. Example arrangements of programming instructions include at least: monolithic structure of instructions; standalone modules of instructions for elements or portions thereof; and/or as modules of instructions that employ external routines, code, services, and so forth; and/or any combination of these, and all such implementations are contemplated to be within the scope of embodiments of the present disclosure Examples of such machines include, without limitation, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements described and/or depicted herein, and/or any other logical components, may be implemented on a machine capable of executing program instructions. Thus, while the foregoing flow charts, block diagrams, and/or operational descriptions set forth functional aspects of the disclosed systems, any arrangement of program instructions implementing these functional aspects are contemplated herein. Similarly, it will be appreciated that the various steps identified and described above may be varied, and that the order of steps may be adapted to particular applications of the techniques disclosed herein. Additionally, any steps or operations may be divided and/or combined in any manner providing similar functionality to the described operations. All such variations and modifications are contemplated in the present disclosure. The methods and/or processes described above, and steps thereof, may be implemented in hardware, program code, instructions, and/or programs or any combination of hardware and methods, program code, instructions, and/or programs suitable for a particular application. Example hardware includes a dedicated computing device or specific computing device, a particular aspect or component of a specific computing device, and/or an arrangement of hardware components and/or logical circuits to perform one or more of the operations of a method and/or system. The processes may be implemented in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer-readable instructions described above. All such permutations and combinations are contemplated in embodiments of the present disclosure.

As described herein, machine learning models may be trained using supervised learning or unsupervised learning. In supervised learning, a model is generated using a set of labeled examples, where each example has corresponding target label(s). In unsupervised learning, the model is generated using unlabeled examples. The collection of examples constructs a dataset, usually referred to as a training dataset. During training, a model is generated using this training data to learn the relationship between examples in the dataset. The training process may include various phases such as: data collection, preprocessing, feature extraction, model training, model evaluation, and model fine-tuning. The data collection phase may include collecting a representative dataset, typically from multiple users, that covers the range of possible scenarios and positions. The preprocessing phase may include cleaning and preparing the examples in the dataset and may include filtering, normalization, and segmentation. The feature extraction phase may include extracting relevant features from examples to capture relevant information for the task. The model training phase may include training a machine learning model on the preprocessed and feature-extracted data. Models may include support vector machines (SVMs), artificial neural networks (ANNs), decision trees, and the like for supervised learning, or autoencoders, Hopfield, restricted Boltzmann machine (RBM), deep belief, Generative Adversarial Networks (GAN), or other networks, or clustering for unsupervised learning. The model evaluation phase may include evaluating the performance of the trained model on a separate validation dataset to ensure that it generalizes well to new and unseen examples. The model fine-tuning may include refining a model by adjusting its parameters, changing the features used, or using a different machine-learning algorithm, based on the results of the evaluation. The process may be iterated until the performance of the model on the validation dataset is satisfactory and the trained model can then be used to make predictions.

In embodiments, trained models may be periodically fine-tuned for specific user groups, applications, and/or tasks. Fine-tuning of an existing model may improve the performance of the model for an application while avoiding completely retraining the model for the application.

In embodiments, fine-tuning a machine learning model may involve adjusting its hyperparameters or architecture to improve its performance for a particular user group or application. The process of fine-tuning may be performed after initial training and evaluation of the model, and it can involve one or more hyperparameter tuning and architectural methods.

Hyperparameter tuning includes adjusting the values of the model's hyperparameters, such as learning rate, regularization strength, or the number of hidden units. This can be done using methods such as grid search, random search, or Bayesian optimization. Architecture modification may include modifying the structure of the model, such as adding or removing layers, changing the activation functions, or altering the connections between neurons, to improve its performance.

Online training of machine learning models includes a process of updating the model as new examples become available, allowing it to adapt to changes in the data distribution over time. In online training, the model is trained incrementally as new data becomes available, allowing it to adapt to changes in the data distribution over time. Online training can also be useful for user groups that have changing usage habits of the stimulation device, allowing the models to be updated in almost real-time.

In embodiments, online training may include adaptive filtering. In adaptive filtering, a machine learning model is trained online to learn the underlying structure of the new examples and remove noise or artifacts from the examples.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### Examples

Materials: Sodium carbonate, lithium bromide, Peroxidase from horseradish Type I (HRP) (P8125), sodium 3,5-dichloro-2-hydroxybenzenesulfonate (B), 4-aminoantipyrine (A), sodium lactate, chitosan, acetic acid, phenol red sodium salt, nitrazine yellow, bromocresol green sodium salt, Whatman™ Grade 1 filter paper and Whatman™ Grade 4 filter paper were purchased from Sigma-Aldrich (USA). Acid Yellow 34 (Y) was purchased from Chem Cruz (USA). Lactate oxidase Grade III (LOx) was purchased from Toyobo (USA). Tegaderm™ Films (size: 4.4×4.4 cm) were purchased from 3M (USA). All chemicals were used as received and they followed trace metal standard, when possible. The chromogenic substrates (i.e., A, B, and Y) were chosen considering their toxicity levels, avoiding the use of harmful or cancerogenic compounds. Silk cocoons of Bombyx mori silkworm were purchased from Tajima Shoji (Japan). Deionized (DI) water with resistivity of 18.2 MΩ cm was obtained with a Milli-Q reagent-grade water system and used to prepare aqueous solutions.

Silk fibroin solution preparation: Silk fibroin was extracted following a previously reported protocol. Briefly, finely chopped Bombyx mori silk cocoons were boiled in a solution of 0.02 M sodium carbonate to remove the sericin layer for 120 minutes. The fibers were washed three times for 20 minutes in DI water, dried overnight, and dissolved in a solution of lithium bromide 9.3 M at 60° C. for 4 hours. The obtained solution was dialyzed against deionized water for 2 days, changing the deionized water 6 times at regular intervals. The final solution was centrifuged twice at a speed of 9000 rpm, at 4° C., for 20 minutes and then filtered yielding 7-8 wt % silk fibroin solution.

Chromogenic enzymatic inks preparation: Silk-based chromogenic enzymatic inks were made of 4 wt % silk fibroin solutions containing 0.1 M PBS as ionic background which keeps the pH constant when reacting with strongly acidic or basic sweat. The final concentration of the enzymatic reagents was 339 U/mL and 150 U/mL for HRP and LOx, respectively. Chromogenic substrates were then dissolved in the silk-based enzymatic mixture to yield final concentrations of: A, 0.86 mg/mL; B, 1.82 mg/mL; Y, 0.3 mg/mL.

Water-based chromogenic enzymatic inks were made of DI water containing 0.1 M PBS as ionic background. The final concentration of the enzymatic reagents was 339 U mL⁻¹ and 150 U mL⁻¹ for HRP and LOx, respectively. Chromogenic substrates were then dissolved in the water-based enzymatic mixture to yield final concentrations of: A, 0.86 mg mL⁻¹; B, 1.82 mg mL⁻¹; Y, 0.3 mg mL⁻¹.

Chromogenic pH-sensitive inks preparation: Silk-based chromogenic pH-sensitive inks were made of 4 wt % silk fibroin solutions containing a pH indicator (i.e., phenol red sodium salt, nitrazine yellow, bromocresol green sodium salt) with a final concentration of 2.5 mg mL⁻¹.

Wearable sensing patches fabrication: The three different paper substrates were cut to obtain squares (side length: 3 cm). 150 μL of chromogenic enzymatic ink were drop-cast in the center of each square and left to dry for 1 h at room temperature. This step was repeated three times to obtain three layers of ink. The functionalized paper was laser-cut to obtain circles (diameter: 3 mm) using a Trotec Speedy 300 Laser Cutter, with 75 W CO₂ laser. The functionalized paper circles were then placed on Tegaderm™ Films to obtain wearable patches.

Functionalization with chitosan: 0.5 w/v % chitosan was dissolved in 2 v/v % acetic acid. To obtain a base layer of chitosan, 150 μL of the solution were drop-cast in the center of each square and left to dry for 1.5 h before the deposition of the three layers of chromogenic enzymatic ink.

Simulated sweat solution preparation: NaCl, KCl, Urea, NH₄Cl, CaCl₂), MgCl₂, were dissolved in DI water to yield final concentrations of 40 mM, 3 mM, 22 mM, 3 mM, 0.4 mM, 50 M respectively.

Analysis of colorimetric response: To induce a colorimetric response, 1 μL of simulated sweat solution at specific concentration of lactate (i.e., 1, 5, 10, 30, 50, 60, 90 mM) was drop-cast on each sensing circle. The fluid spread on the surface of the sensing circle and mixed with the reagents to produce a colorimetric response. The colorimetric response was analyzed collecting images using a Laser Jet Pro MFP M127fn scanner from HP (USA), 24-bit color depth and resolution of 600 dpi. ImageJ was used to quantify the response as variations in the Red, Green or Blue channel intensities. The specificity was evaluated by measuring the variation in the sensors' colorimetric response in the Green Channel before and after the exposure to interfering substances (NaCl, KCl, Urea, NH₄Cl, CaCl₂), MgCl₂, with concentrations of 40 mM, 3 mM, 22 mM, 3 mM, 0.4 mM, 50 M respectively):

$$Green Variation ( \% ) = \frac{{G}_{before} - {G}_{after exposure}}{{G}_{before}}$$

Accelerated degradation tests: Accelerated degradation tests were performed on lactate sensing patches fabricated with silk-based or water-based chromogenic enzymatic ink, both with or without a base layer of chitosan. The patches were stored at 60° C., above the enzyme degradation temperature, for 8, 24, 120 hours and 2.5 months. After storage, the colorimetric response was analyzed to assess the ability of silk-based inks to maintain enzymatic activities when exposed to lactate variations in the 0-90 mM range. To evaluate the long-term stability, the patches were stored at 4° C. for 18, 21 and 24 months. After storage, the colorimetric response was analyzed to assess the ability of silk-based inks to maintain enzymatic activities when exposed to lactate variations in the 0-90 mM range. The retained activity was evaluated by measuring the variation in the sensors' colorimetric response at 90 mM (in the Green Channel) right after fabrication and after storage:

$$Retained Activity ( \% ) = \frac{{G}_{90 mM} ( t = storage time ) - {G}_{0 mM} ( t = storage time )}{{G}_{90 mM} ( t = 0 ) - {G}_{0 mM} ( t = 0 )}$$

Neural Network Training: Wearable sensing patches for neural network training were made as follows. Whatman™ Grade 1 filter paper was functionalized with a base layer of chitosan solution and three layers of silk-based chromogenic enzymatic ink. The paper was laser-cut to obtain circles (diameter: 3 mm) using a Trotec Speedy 300 Laser Cutter, with 75 W CO2 laser. Four sensing paper circles were applied on Tegaderm™ Films. To allow easier machine learning-driven lactate concentration prediction, four reference colors (RGB values: red (255, 0, 0), green (0, 255, 0), blue (0, 0, 255) and light yellow (253, 252, 188)) were included in the form of non-sensing colored paper circles. The reference colors were printed on Whatman™ Grade 1 filter paper using a Laser Jet Pro MFP M127fn printer from HP (USA), 24-bit color depth and resolution of 600 dpi. The non-sensing colored paper circles (diameter: 3 mm) were laser-cut using a Trotec Speedy 300 Laser Cutter, with 75 W CO₂ laser and applied on Tegaderm™ Films where the sensing circles were previously applied.

Image acquisition: 1 μL of simulated sweat solution containing a precise amount of lactate at specific concentration of lactate (i.e., 1, 5, 10, 30, 50 mM) was drop-cast on each sensing circle. After the colorimetric response, images of the sensors were acquired in different light conditions using a smartphone camera (Apple, iPhone SE 2020). The images were used to train the neural network and to evaluate its performance. The trained neural network was able to predict the lactate concentration when an image of the sensor was given as input.

Image Pre-processing: Computer Vision libraries and Machine Learning algorithms were used to allow quantitative readouts. The lightness and warmth level of the acquired images were standardized using reference images (i.e., images acquired in controlled light conditions to avoid shadows and differences in warmth level that could affect the analysis of the colorimetric response of the sensing circles). The brightness of the acquired images was adjusted to match the reference images in the HSV (Hue, Saturation, and Value of brightness) color space to obtain uniform light conditions in the datasets. Afterwards, using the CIELAB (International Commission on Illumination L*a*b*, L*-Lightness, a*-redness, b*-yellowness) color space, the average b* values of the background color of the reference image were extracted and set as standard values. The b* values of all the images were shifted to match these standard values, while the L* values were set to 130 to avoid lightness differences that could affect the colorimetric analysis. Finally, a color-based image filter was used to select only pixels corresponding to the sensing circles of the patches. The OpenCV and Pillow libraries were used to convert images through different color spaces and to create masks for color extraction.

Machine Learning: The data was analyzed to determine the best model category for the colorimetric analysis of the lactate-sensing patches. After early data exploration, different Support Vector Machine (SVM) models were trained for the multi-class classification. The model building was developed using RStudio and the package “e1071” for SVM models.

The SVM model was trained with three different datasets (DS1, DS2, DS3) containing pictures of the lactate-sensing patches applied on the skin of three different people (DS1, n=955; DS2, n=594; DS3, n=665 images). The datasets were studied either analyzing 6 predicted lactate concentrations (i.e., 0 mM, 1 mM, 5 mM, 10 mM, 30 mM, 50 mM), or analyzing 4 predicted classes of lactate concentration (i.e., No lactate=0 mM, Low concentration=1 mM, Medium concentration 5-10 mM, and High concentration 30-50 mM). Each dataset was divided into six categories corresponding to the different lactate concentrations, leading to three different datasets. To have a larger and more heterogeneous dataset, Dataset 1, Dataset 2, and Dataset 3 were merged to obtain the full dataset (n=1880, Dataset 4). The values of sensitivity (True Positive Rate), specificity (True Negative Rate), Positive Predicted Value (PPV) and Negative Predicted Value (NPV) were similar across all datasets at each lactate concentration or class of lactate concentrations evaluated. In general, the values were highest at the lowest concentrations of lactate evaluated (e.g., 0 mM, 1 mM). Additional data are provided in U.S. Application Ser. Nos. 63/483,959 and 63/512,534, Matzeu, G., et al. (2020). Large-Scale Patterning of Reactive Surfaces for Wearable and Environmentally Deployable Sensors. Advanced Materials, 32(28), 2001258; and Ruggeri, E., et al. (2023). Paper-Based Wearable Patches for Real-Time, Quantitative Lactate Monitoring. Advanced Sensor Research, all of which are incorporated by reference herein in their entirety for all purposes.

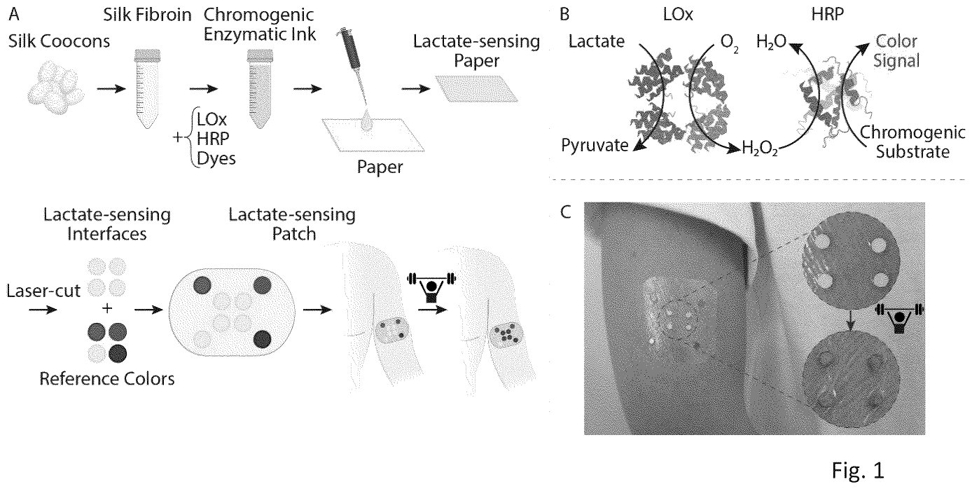

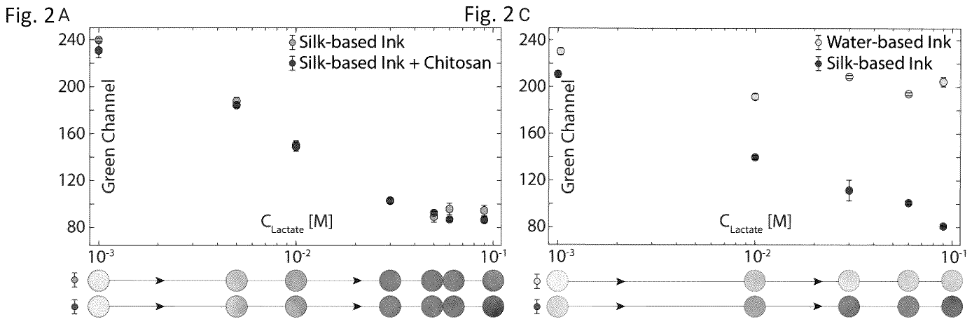

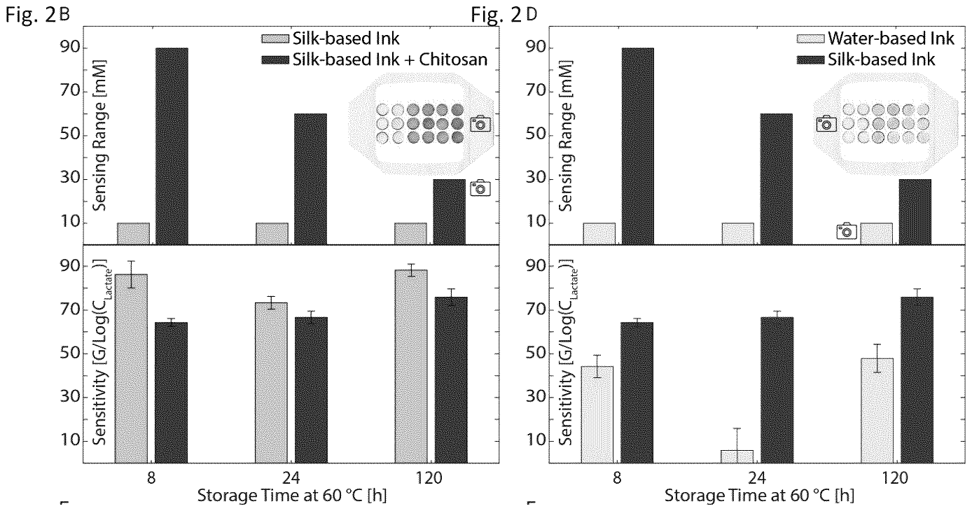

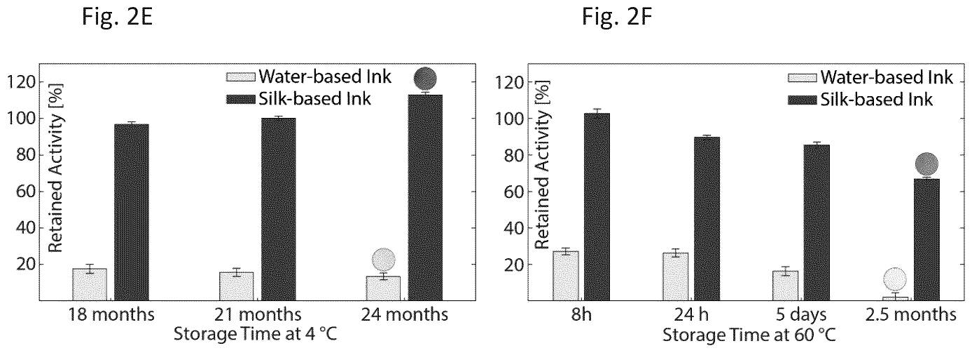

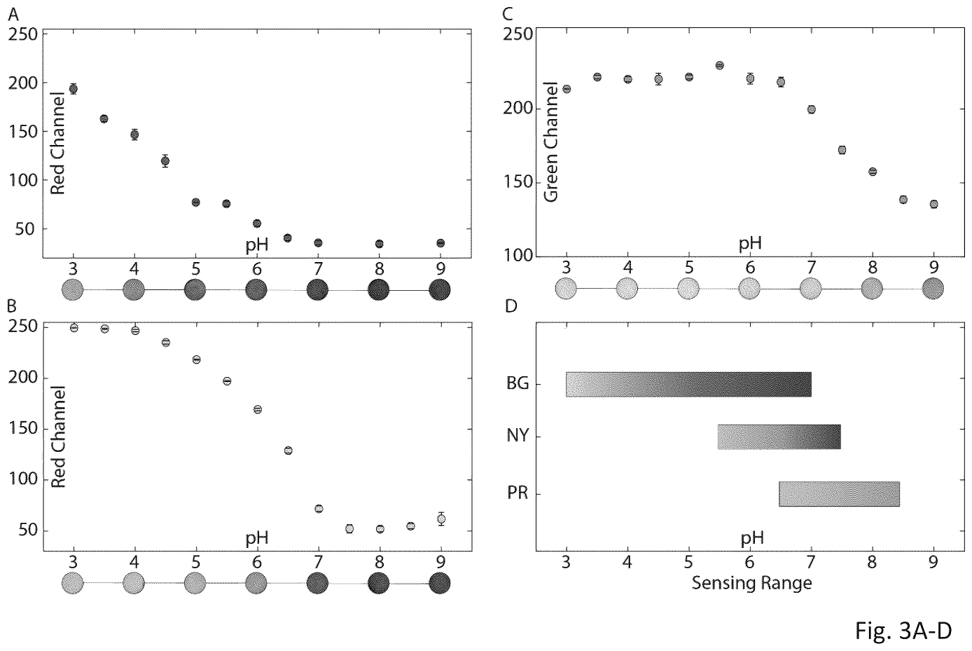

Colorimetric wearable lactate-sensing patches: The silk-based chromogenic enzymatic inks were formulated by incorporating lactate oxidase (LOx) and horseradish peroxidase (HRP) with the chromogenic substrates in a regenerated silk fibroin aqueous solution that enabled the stabilization of the labile molecules. The biosensing composite ink was infiltrated in small (i.e., 3 mm diameter) filter paper discs (Error! Reference source not found.a) which are then arranged in pre-specified geometries onto a Tegaderm™ wound dressing film. Additional non-reactive inks containing reference dyes are added to the construct to produce the final wearable patches. These are flexible, conformable and can be worn for several hours without causing discomfort. The use of a semipermeable wound dressing film allows for natural breathing of the skin—being permeable to water vapor, oxygen, and carbon dioxide—while ensuring contact with the absorbing bioresponsive discs for sweat collection (FIG. 1c).

Upon contact with sweat, the lactate-responsive discs change color from yellow to dark red as a function of lactate concentration, while the nonreactive reference circles provide fiducial markers to correct for lighting artifacts and boundary conditions for the machine-learning driven image processing stage. The colorimetric response of the composite inks follows the LOx/HRP cascade reaction (Error! Reference source not found.b) where LOx oxidizes lactate to produce pyruvate and hydrogen peroxide, which is then used by HRP to oxidize the chromogenic substrates generating an immediately visible color change.

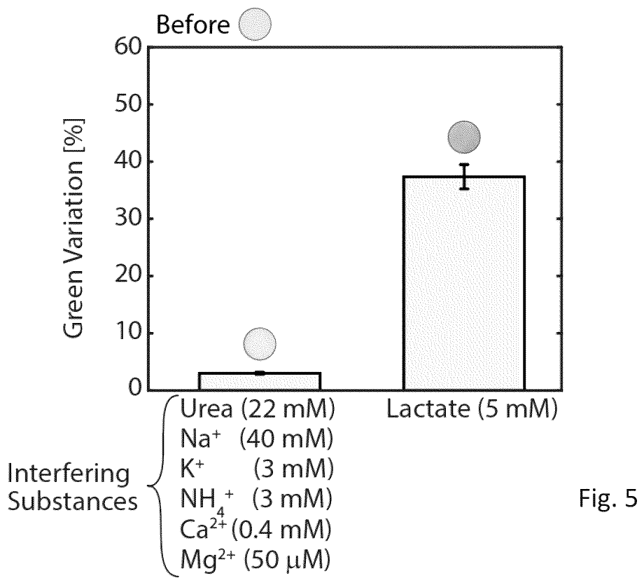

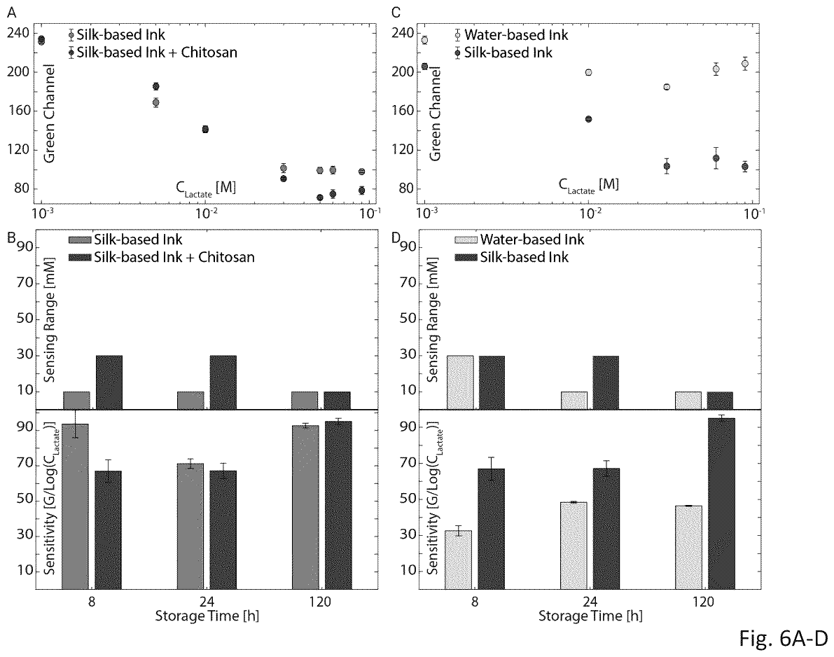

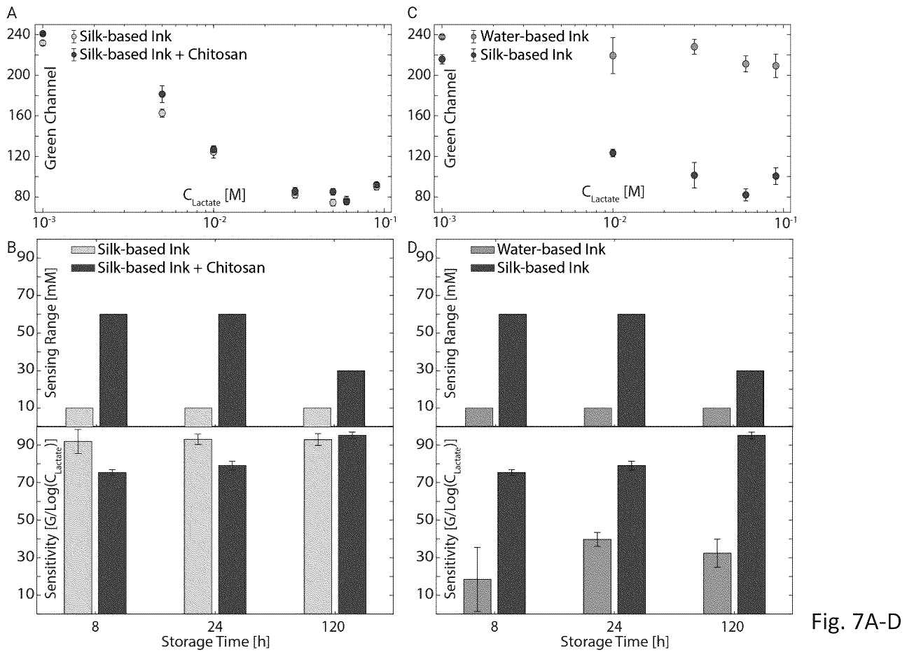

Colorimetric response evaluation: Despite the effectiveness of colorimetric sensing techniques for rapid detection of analytes, the reproducibility of these systems is compromised by the potential presence of color gradients in the sensing areas. The lack of color uniformity compromises the readout reliability especially if coupled with image recognition models to obtain quantitative results. The color gradient, namely coffee-ring effect, in microfluidic paper-based sensors is attributed to the sample solution transporting the chromogenic substrates and the enzymes while diffusing from the center of the sensing areas to the edges, resulting in a heterogeneous coloration. To avoid this problem, the paper substrate was modified with a chitosan solution which bonds to paper through electrostatic interactions and forms a thin film on the porous paper structure resulting in the immobilization and adhesion of the ink components. The effect of the deposition of a base layer of chitosan on the performances of the silk-based ink was investigated using Whatman grade 1 filter paper (Error! Reference source not found.a). First, the sensing interfaces were calibrated using solutions that mimic sweat composition with lactate concentrations in the 0-90 mM range to assess the effect of chitosan on the colorimetric response. The sensitivity and sensing range were calculated by evaluating the slope of the calibration curve (i.e., Green channel (G) as a function of the logarithm of lactate concentration (Log_{C})). The presence of the base layer of chitosan yielded an increased sensing range (with chitosan, 0-90 mM; without chitosan, 0-60 mM) without compromising the sensitivity (with chitosan, −84.11±3.31 G Log_{C}⁻¹; without chitosan, −87.29±3.77 G Log_{C}⁻¹ in the 0-60 mM range; avg±s.e., n=5) and improved the reproducibility of the colorimetric response at high lactate concentration (coefficient of variation in the 50-90 mM range: with chitosan, 4.62±0.68%; without chitosan, 11.34±1.97%; avg±s.e.) proving its efficacy in improving the colorimetric response. The combination of such high sensitivity and wide linear sensing range is of high utility for the colorimetric detection of lactate, which is commonly reported to have an upper detection limit of 25 mM (i.e., lower than the concentration required for the analysis of undiluted sweat). The colorimetric sensor presented in this study has a wide linear sensing range (e.g., 0-90 mM) suitable for both training monitoring and disease diagnostic. Specifically, for sport medicine applications lactate concentrations in undiluted sweat can reach 60 mM and further increase during training while for clinical applications, a upper detection limit of 50 mM may be needed. The specificity of the sensor was demonstrated by evaluating the absence of colorimetric response after the exposure to interfering substances (i.e., urea, sodium, potassium, ammonium, calcium, magnesium ions)(FIG. 5). The sensing performances of Ahlstrom grade 55 filter paper and Whatman grade 4 filter paper were also evaluated (FIG. 6 and FIG. 7). Whatman grade 1 filter paper showed the best performances among the three types of paper. The difference in colorimetric response is attributed to the pore size of the paper: Whatman grade 1 has the smallest pore size (i.e., 11 μm), compared to Ahlstrom grade 55 and Whatman grade 4 (i.e., 15 and 20-25 μm respectively), which decreases the transport of the chromogenic substrates and the enzymes to the edges of the sensing areas increasing both the color homogeneity and reproducibility.

Shelf-life evaluation: The commercialization of enzymatic sensors has been set back by the progressive decrease in stability of the enzymes and dried proteins during storage at room temperature. Despite representing their main drawback, this problem has not been addressed by previous studies and stability tests are often lacking. Here, the demonstrated ability of regenerated silk-fibroin to stabilize labile biomolecules is exploited to improve the thermal stability of the sensors. In fact, encapsulating enzymes in a silk fibroin matrix delays the degradation of their native structure by reducing their molecular mobility and providing protection against environmental factors such as temperature and pH changes through a buffering action. To prove the prolonged shelf-life of the sensing interfaces both with and without the base layer of chitosan, the sensors were subjected to accelerated degradation tests by storing them at 60° C. (i.e., above the degradation temperature of the enzymes) for 8, 24 and 120 h. The ability of the inks to preserve enzymatic activity was assessed when exposed to lactate variations (i.e., 0-90 mM). Storage for 8 h at 60° C. did not affect the sensing range of the sensing interfaces with chitosan, which showed a linear response in the 0-90 mM range. The sensing range without chitosan was reduced to 0-30 mM. Chitosan did not affect the sensitivity in the 0-30 mM range (with chitosan, −67.73±5.86 G Log_{C}⁻¹; without chitosan, −72.21±3.75 G Log_{C}⁻¹; avg±s.e., n=3). When increasing the storage time to 24 and 120 h at 60° C., the presence of chitosan caused a slower reduction of the sensing range (with chitosan: 24 h, 0-60 mM; 120 h, 0-30 mM; without chitosan: 24 h, 0-10 mM, 120 h, 0-10 mM), which further confirms the combined ability of silk and chitosan to improve the colorimetric response. The key role of silk fibroin in the stabilization of the enzymes was demonstrated by comparing the sensing performances of silk-based and water-based inks (both in the presence of a base layer of chitosan) when subjected to accelerated degradation tests (i.e., storing them at 60° C., above the degradation temperature of the enzymes for 8, 24 and 120 h). The colorimetric response of the sensing interfaces was evaluated after 8 h of storage (Error! Reference source not found.c). As opposed to the silk-based interfaces, water-based interfaces have a narrower sensing range (water-based: 0-10 mM; silk-based: 0-90 mM) and lower sensitivity in the 0-10 mM range (water-based: −44.24±5.18 G Log_{C}¹; silk-based −70.96±2.32 G Log_{C}⁻¹; avg±s.e., n=3) (Error! Reference source not found.d). Additionally, the colorimetric response of the silk-based interfaces is stronger (i.e., dark red) than for the water-based ones (i.e., light pink) (Error! Reference source not found.c, insets). The sensing performances of Ahlstrom grade 55 filter paper and Whatman grade 4 filter paper were also evaluated after accelerated degradation tests (FIG. 6 and FIG. 7). Also in this case, Whatman grade 1 filter paper showed the best performances among the three studied papers. The stability of these silk-based sensors is remarkable since, even after 8, 24 and 120 h at 60° C., they reach a higher upper detection limit (90 mM, 60 mM and 30 mM, respectively) compared to previously reported colorimetric sensors for lactate detection. The long-term stability of the wearable sensors was evaluated after 18, 21 and 24 months of storage at 4° C. (Error! Reference source not found.e) and 2.5 months of storage at 60° C. (Error! Reference source not found.f). The silk-based chromogenic enzymatic mixture was able to preserve 66% of its activity after 2.5 months at 60° C., as opposed to water-based control whose activity decreased to 2%. Additionally, when stored at 4° C., the silk-based chromogenic enzymatic mixture completely retained its activity for up to 24 months at 4° C., while in the water-based control the activity was reduced to 17%. These results further demonstrate the ability of silk fibroin to stabilize the chromogenic enzymatic mixture allowing the fabrication of shelf-stable sensors whose colorimetric response is not undermined after years of storage.

Colorimetric wearable pH-sensing patches: The versatility of this approach and the possibility to develop multi-sensing patches, was demonstrated by developing silk-based chromogenic inks incorporating pH-responsive molecules (i.e., nitrazine yellow (NY), bromocresol green (BG), and phenol red (PR)). The inks presented in this study show high sensitivity (i.e., BG, −39.8±1.3; NY, −76.1±1.4; PR, −40.9±1.9; avg±s.e., n=3) and reversibility suitable for the detection of pH variations in real-time. The intensity of the colorimetric response in the RGB color space for each ink was evaluated (Error! Reference source not found.). The three different inks have three complementary sensing ranges (i.e., BG, pH range 3-7; NY, pH range 5.5-7.5; PR, pH range 6.5-8.5) and, by combining them on the same sensing patch, it is possible to read the pH value in the physiologically relevant sweat pH range (i.e, pH 3-8.5 pH). Together with lactate, sweat pH is an important variable for health monitoring due to its correlation with sodium concentration which makes it an indicator of dehydration. The application of both the lactate and pH silk-based sensing interface on the same patch yields shelf-stable multianalyte sensors for comprehensive health monitoring.

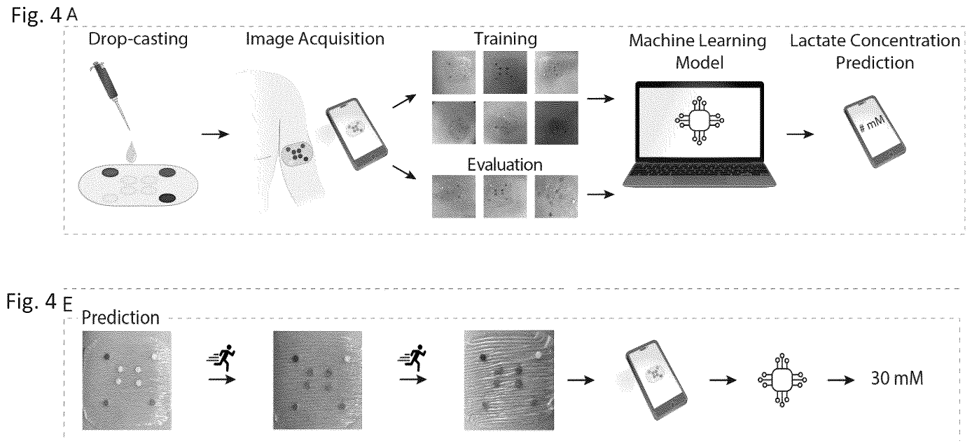

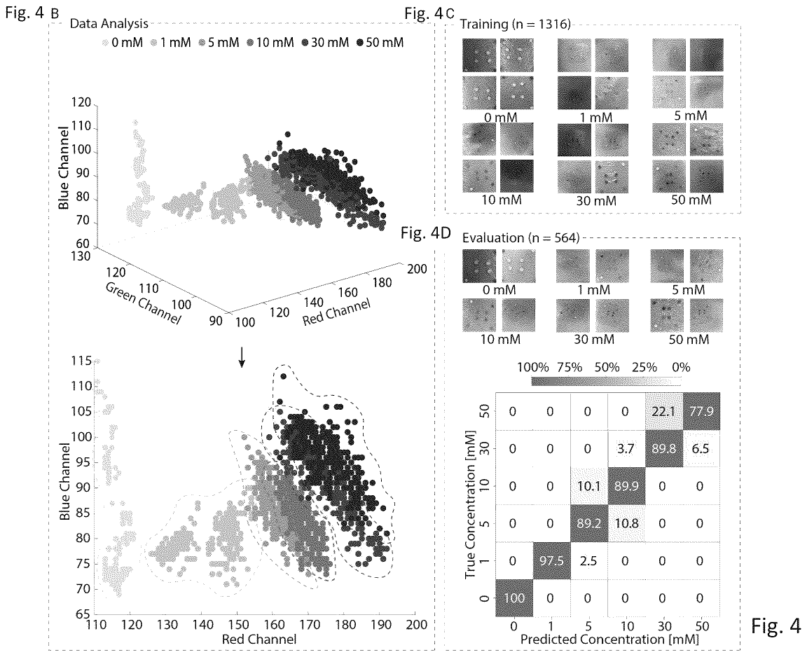

Machine learning-driven readout: To allow easy quantitative readouts of the colorimetric response using a smartphone camera, 1961 images of the sensors at 6 different concentrations of lactate (i.e., 0, 1, 5, 10, 30, 50 mM) were obtained and used to develop a machine learning model. The images were acquired after drop-casting the simulated sweat solution with a known lactate concentration on the sensing interfaces of the sensors (Error! Reference source not found.a). To obtain a universal model able to recognize images under non-ideal light exposure, the images were acquired under different light conditions and then randomly divided in two groups: 1373 labeled images were used for the model training (Error! Reference source not found.c) and 588 were used for the evaluations of its performances (Error! Reference source not found.d (top)). The images of the sensors were acquired over three different days that corresponded to three different datasets, the combination of the three was analyzed as a fourth dataset (i.e., full dataset).A Support Vector Machine (SVM) model was built to read and predict the colorimetric response of the sensors as a function of lactate concentration. Plots of the RGB values extracted from each dataset show that it is possible to clearly identify six data clusters (i.e., one for each lactate concentration). The clusters have a low standard deviation (i.e., 0.92-4.13) and their organization in a three-dimensional space enables the identification of separatory hyperplanes suitable to build a SVM predictive model (Error! Reference source not found.b). Given the considerations above, the SVM model was found to be ideal for this system based on early data exploration during which the colorimetric variation of the sensors at different lactate concentrations was analyzed for each dataset. The increase in lactate concentration produced a color change from yellow to red, which, at the image analysis stage, corresponds to a decrease of the value of the green channel in the RGB color space. The analysis reveals a narrow data distribution for each lactate concentration, demonstrating the ability of the model to take into account and correct for different light conditions which, if not accounted for, would cause low prediction accuracy for colorimetric sensors. The predictive model was built for each dataset, using the 70% of the images in each dataset for the model training. The remaining 30% of the images in the datasets was used as an input to evaluate how accurately the SVM model classifies the sensor images. To calculate the accuracy of the model, the confusion matrix—whose diagonal shows the percentage of correctly classified images—was evaluated for the full dataset (FIG. 4d (bottom)). The model was able to reach an overall accuracy of 93%, 96% and 92% for datasets 1, 2 and 3 respectively, which proved the excellent predictive capabilities of the model. To show the classification accuracy of the model on a big and heterogenous dataset, the model was built and trained on the full dataset (namely dataset 4) and reached an overall accuracy of 89.3%.

After obtaining high prediction accuracy using a quantitative classification of the images, an additional model was built using a qualitative classification by dividing each dataset into four categories (i.e., no lactate, low concentration, medium concentration, high concentration). The suitability of the SVM model for this type of classification was confirmed and an evaluation of its classification accuracy revealed a high prediction accuracy. Similarly, the model built on the full dataset presented high performance with an overall accuracy of 98.8%, showing the ability to obtain accurate readouts. Finally, the feasibility of the application of machine-learning driven quantitative readouts in a real-life scenario was demonstrated by placing the wearable sensors on the skin of a volunteer during a treadmill exercise session. The image of the wearable sensors after the session was acquired and processed by the SVM model which predicted a 30 mM lactate concentration (FIG. 4e).

1. A printable liquid lactate sensor composition, the composition comprising:
silk fibroin in an amount by weight of between 0.1% and 30%;
a lactate oxidase that is activated by lactate to produce hydrogen peroxide;
a peroxidase that is activated by the hydrogen peroxide; and
a chromogenic substrate that changes color upon activation of the peroxidase.

2. A solid sensor comprising a biopolymer substrate formed from the printable liquid lactate sensor composition of claim 1, the biopolymer substrate having embedded therein the lactate oxidase, the peroxidase, and the chromogenic substrate,
wherein a quantitative bulk colorimetric change of the biopolymer substrate varies as a lactate concentration within the biopolymer substrate varies from 0.1 mM to 100 mM.

3. A wearable sensor for detecting lactate, the wearable sensor comprising:
a biopolymer substrate having embedded therein a lactate oxidase that is activated by the lactate to produce hydrogen peroxide, a peroxidase that is activated by the hydrogen peroxide, and a chromogenic substrate that changes color upon activation of the peroxidase, the biopolymer substrate comprising silk fibroin in an amount by weight of between 1% and 100%,
wherein a quantitative bulk colorimetric change of the biopolymer substrate varies as a lactate concentration within the biopolymer substrate varies from 0.1 mM to 100 mM.

4. A sweat sensor comprising:
a biopolymer substrate having embedded therein a lactate oxidase that is activated by lactate to produce hydrogen peroxide, a peroxidase that is activated by the hydrogen peroxide, and a chromogenic substrate that changes color upon activation of the peroxidase, the biopolymer substrate comprising silk fibroin in an amount by weight of between 1% and 100%,
wherein a quantitative bulk colorimetric change of the biopolymer substrate varies as a lactate concentration within the biopolymer substrate varies from 0.1 mM to 100 mM.

5. The printable liquid lactate sensor composition of claim 1, wherein the chromogenic substrate comprises a baseline colorant.

6. The printable liquid lactate sensor composition of claim 5, wherein the baseline colorant is a yellow dye.

7. The printable liquid lactate sensor composition of claim 6, wherein the yellow dye is an acid yellow dye.

8. The printable liquid lactate sensor composition of claim 7, wherein the acid yellow dye is Acid Yellow 34.

9. The printable liquid lactate sensor composition of claim 1, wherein the chromogenic substrate comprises sodium 3,5-dichloro-2-hydroxygenzenesulfonate and/or 4-aminoantipyrine.

10. The printable liquid lactate sensor composition of claim 1, wherein the printable liquid lactate sensor composition comprises a baseline buffer and/or electrolyte mixture.

11. The solid sensor of claim 2, wherein the biopolymer substrate comprises a base layer of chitosan.

12. The solid sensor of claim 2, wherein the quantitative bulk colorimetric change occurs substantially immediately upon contact with a solution having a lactate concentration of at least 0.1 mM.

13. An article of clothing comprising a plurality of the solid sensor of claim 2.

14. A wearable patch comprising a plurality of the solid sensor of claim 2.

15. The article of clothing of claim 13, further comprising reference color spots having predetermined known colors for colorimetric analysis of images of the solid sensor or solid sensors.

16.-46. (canceled)

47. The wearable patch of claim 14, further comprising reference color spots having predetermined known colors for colorimetric analysis of images of the solid sensor or solid sensors.

48. The printable liquid lactate sensor composition of claim 10, wherein the baseline buffer and/or electrolyte mixture is tailored to mimic human sweat.

49. The printable liquid lactate sensor composition of claim 1, wherein the peroxidase is horseradish peroxidase (HRP).

50. The wearable sensor of claim 3, wherein the chromogenic substrate comprises a baseline colorant.

51. The sweat sensor of claim 4, wherein the chromogenic substrate comprises a baseline colorant.
